(19) United States
(12) Patent Application Publication
(10) Pub. No.: US 2005/0278011 A1
(43) Pub. Date: Dec. 15, 2005

(54) Stent delivery system

(76) Inventor: John E. Peckham, Sunnyvale, CA (US)

Correspondence Address: VIDAS, ARRETT & STEINKRAUS, P.A., 6109 BLUE CIRCLE DRIVE, SUITE 2000, MINNETONKA, MN, 55343-9185, US

(21) Appl. No.: 10/864,989

(22) Filed: Jun. 10, 2004

(51) Int. Cl. A61F 2/06

(52) U.S. Cl. 623/1.11

(57) ABSTRACT

A medical device comprises a catheter having a balloon positioned thereon, a stent and a sheath. The sheath is disposed about the stent when the stent is in a reduced diameter configuration. When the stent is in the reduced diameter configuration it is disposed about a portion of the catheter distally adjacent to the balloon. The catheter is moveable relative to the sheath.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

1. Field of the Invention

This invention relates to catheters and catheter assemblies for use in medical procedures. More specifically, this invention relates to a stent delivery catheter system, such as the kind used in percutaneous transluminal coronary angioplasty (PTCA) procedures, intracranial aneurysm stenting and/or other systems for the delivery of a stent into a body lumen.

2. Description of the Related Art

Percutaneous transluminal coronary angioplasty (PTCA) is a procedure which is well established for the treatment of blockages, lesions, stenosis, thrombus, etc. present in body lumens such as the coronary arteries and/or other vessels.

A widely used form of percutaneous coronary angioplasty makes use of a dilatation balloon catheter which is introduced into and advanced through a lumen or body vessel until the distal end thereof is at a desired location in the vasculature. Once in position across a afflicted site, the expandable portion of the catheter, or balloon, is inflated to a predetermined size with a fluid at relatively high pressures. By doing so the vessel is dilated, thereby radially compressing the atherosclerotic plaque of any lesion present against the inside of the artery wall, and/or otherwise treating the afflicted area of the vessel. The balloon is then deflated to a small profile so that the dilatation catheter may be withdrawn from the patient's vasculature and blood flow resumed through the dilated artery.

In angioplasty procedures of the kind described above, there may be restenosis of the artery, which either necessitates another angioplasty procedure, a surgical by-pass operation, or some method of repairing or strengthening the area. To reduce restenosis and strengthen the area, a physician can implant an intravascular prosthesis for maintaining vascular patency, such as a stent, inside the artery at the lesion.

Stents, grafts, stent-grafts, vena cava filters, expandable frameworks, and similar implantable medical devices, collectively referred to hereinafter as stents, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, etc. Stents may be used to reinforce body vessels and to prevent restenosis following angioplasty in the vascular system. They may be self-expanding, such as a nitinol shape memory stent, mechanically expandable, such as a balloon expandable stent, or hybrid expandable.

Prior to delivery a stent or stents may be retained on a portion of the delivery catheter by crimping the stent onto the catheter, retaining the stent in a reduced state about the catheter with a removable sheath, sleeve, sock or other member or members, or by any of a variety of retaining mechanisms or methods. Some examples of stent retaining mechanisms are described in U.S. Pat. No. 5,681,345; U.S. Pat. No. 5,788,707; U.S. Pat. No. 6,066,155; U.S. Pat. No. 6,096,045; U.S. Pat. No. 6,221,097; U.S. Pat. No. 6,331,186; U.S. Pat. No. 6,342,066; U.S. Pat. No. 6,350,277; U.S. Pat. No. 6,443,880; U.S. Pat. No. 6,478,814 and U.S. patent application Ser. No. 09/664,268 entitled Rolling Socks and filed Sep. 18, 2000.

It is known that in some stent delivery catheters, a balloon expandable stent is disposed about the balloon prior to delivery. In some cases a retractable sleeve or other member may be disposed about the stent to protect the stent during advancement of the catheter. Often, such as in the case of self-expanding stents, the catheter may avoid the use of a balloon, thus allowing the catheter to attain a lower profile. In some cases however, the self-expanding stent will need to be “seated” or otherwise pushed against the body lumen or vessel into which it has expanded in order to properly secure the stent in place. In such instances a separate balloon catheter is often advanced to the cite of the expanded stent, wherein the balloon is expanded to properly seat the stent.

Thus, a need exists to provide for a stent delivery system which has the capability to both deliver and seat a self-expanding stent while also providing the catheter with a desired low profile prior to delivery.

All U.S. patents, applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### BRIEF SUMMARY OF THE INVENTION

The present invention is embodied in a variety of different forms. For example, in at least one embodiment the invention is directed to a low profile stent delivery system. In some embodiments the invention is directed to a catheter assembly for delivery of a self-expanding stent wherein prior to delivery, the stent is mounted distally of a balloon on the catheter. In some embodiments the assembly comprises an outer housing or sheath which contains the stent and balloon prior to deliver of the stent. In some embodiments at least a portion of the catheter shaft is moveable relative to the sheath. In some embodiments the distal waist of the balloon is mounted to an inner shaft of the catheter and the proximal waist is mounted to an outer shaft of the catheter; an inflation lumen, in fluid communication with the balloon is defined between the inner and outer shafts. In at least one embodiment the inner shaft defines a guidewire lumen.

In some embodiments the sheath retains the stent in an unexpanded or predelivery state, such that the reduced diameter of the stent is maintained within the inner diameter of the sheath. The outer diameter of the balloon in the unexpanded or predelivery configuration is sufficient to abut the proximal edge of the stent and otherwise occlude the space immediately proximal of the stent between the sheath and the inner shaft. As a result, prior to delivery of the stent the balloon acts to prevent unintended proximal migration of the stent relative to the catheter shaft and/or sheath. In addition, the balloon may also function as a push device for advancing the stent distally out of the outer housing, whereupon the stent will self-expand. Subsequent to delivery, the same balloon may be advanced out of the sheath and through the stent whereupon it may be expanded in order to seat the stent if necessary.

In some embodiments the catheter assembly comprises one or more hubs, rings and/or other members for engaging stent prior to and/or during advancement of the catheter through the outer sheath.

In some embodiments the assembly comprises one or more areas, bands, coatings, members, etc. that is (are) radiopaque or otherwise detectable by imaging modalities such as X-Ray, MRI or ultrasound. In at least one embodiment one or more marker bands are positioned on the catheter shaft and/or outer sheath adjacent to the balloon and/or the stent.

In the various embodiments, the stent is a self-expanding stent. As such the stent may be constructed of a variety of substances which will allow the stent to exhibit self-expansion characteristics. In some embodiments the stent is at least partially constructed of any of a variety of materials such as stainless steel, nickel, titanium, nitinol, platinum, gold, chrome, cobalt, as well as any other metals and their combinations or alloys. In some embodiments the stent may be at least partially constructed of a polymer material. In some embodiments the stent may be at least partially constructed of a shape-memory polymer or material. In some embodiments the stent may be self-expandable, or hybrid expandable. In some embodiments a stent may include one or more radiopaque members. In some embodiments a stent may include one or more therapeutic and/or lubricious coatings applied thereto.

These and other embodiments which characterize the invention are pointed out with particularity in the claims annexed hereto and forming a part hereof. However, for a better understanding of the invention, its advantages and objectives obtained by its use, reference should be made to the drawings which form a further part hereof and the accompanying descriptive matter, in which there is illustrated and described a embodiments of the invention.

### BRIEF DESCRIPTION OF THE SEVERAL VIEWS OF THE DRAWING(S)

A detailed description of the invention is hereafter described with specific reference being made to the drawings.

FIG. 1 is a longitudinal cross-sectional view of an embodiment of the invention shown prior to delivery of the stent.

FIG. 2 is a longitudinal cross-sectional view of the embodiment depicted in FIG. 1 shown during delivery of the stent from the catheter.

FIG. 3 is a longitudinal cross-sectional view of the embodiment depicted in FIG. 2 shown after the stent has been released from the catheter.

FIG. 4 is a longitudinal cross-sectional view of the embodiment depicted in FIG. 3 wherein the balloon is shown expanded to seat the stent.

FIG. 5 is a longitudinal cross-sectional view of the embodiment depicted in FIG. 1 wherein the distal balloon waist and/or cone comprises a reinforced region.

FIG. 6 is a longitudinal cross-sectional view of the embodiment depicted in FIG. 1 wherein the inner catheter shaft comprises one or more stent engagement members distal of the balloon.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

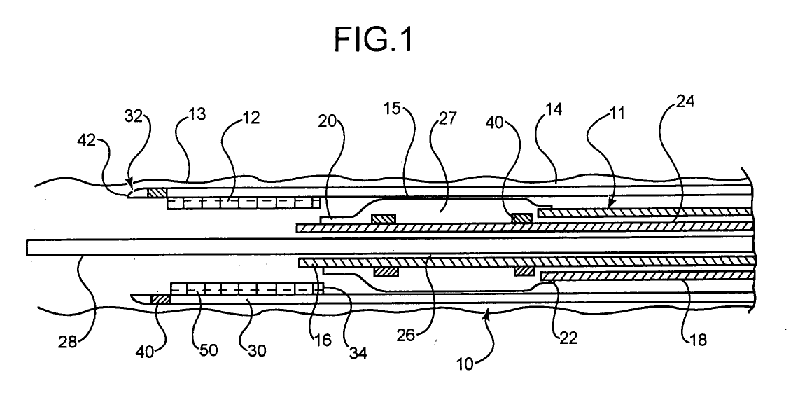

The present invention is embodied in a variety of forms. In at least one embodiment, an example of which is shown in FIG. 1, the invention is directed to a medical device 10 for deployment of a stent, such as a self-expanding stent 12 into a body lumen or vessel 14.

Device 10 comprises a catheter 11 having an inner shaft 16 and an outer shaft 18. The distal waist 20 of the balloon 15 is engaged to the inner shaft 16. The proximal waist 22 of the balloon 15 is engaged to the outer shaft 18. The inner shaft 16 and the outer shaft 18 define an inflation lumen 24 therebetween. The inflation lumen 24 is in fluid communication with the interior 27 of the balloon 15. It should be noted however, that various shaft, balloon, and lumen configurations may be used in the present invention. For example in some embodiments the balloon may be engaged at both end to a single inner shaft. The shaft may define an inflation lumen which communicates with the balloon interior.

In at least one embodiment the inner shaft 16 defines a guidewire lumen 26 through which a guidewire 28 is positioned. The assembly 10 may be advanced along the guidewire 28 to a predetermined location within the vessel 14. Alternatively, the catheter assembly may be configured as a push catheter, thus avoiding the need for the guidewire and guidewire lumen.

Disposed about the catheter 11 is an outer housing or sheath 30. The catheter 11 is independently moveable relative to the sheath 30. A distal end region 32 of the sheath is disposed about the stent 12 and acts to retain the self-expanding stent 12 in a reduced or pre-delivery state. The outer sheath 30 is withdrawn to release the stent 12. During withdrawal of the outer sheath 30, at least a portion of the balloon 15, such as for example the distal waist 20, acts to lock the stent 12 in place while the outer sheath 30 is retracted.

In at least one embodiment, the outer sheath 30 is at least partially constructed from a material having a sufficient hoop strength to retain the stent 12 in the reduced or predelivery diameter. Some examples of suitable material(s) which the sheath 30 may be constructed include but are not limited to: pebax, polyimide, nylon, polyethylene, high density polyethylene (HDPE) etc. The material of the sheath 30 may be a single material or a composite of multiple materials. For example in at least one embodiment the sheath 30 comprises a layer of HDPE with a coil of Pebax or similar material disposed thereabout. In at least one embodiment one or more materials of the sheath 30 may comprise a braided configuration.

As shown in FIG. 1, the stent 12 is retained by the sheath 30 and is positioned distally adjacent to the balloon 15. The balloon 15, like the stent 12 is limited in diameter by the presence of the sheath 30 prior to delivery. Within the confines of the sheath 30 the balloon has a nominal outer diameter sufficient to abut the proximal edge 34 of the stent 12.

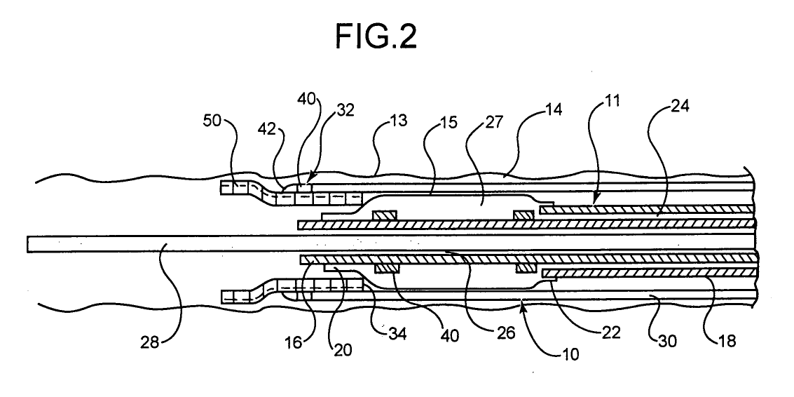

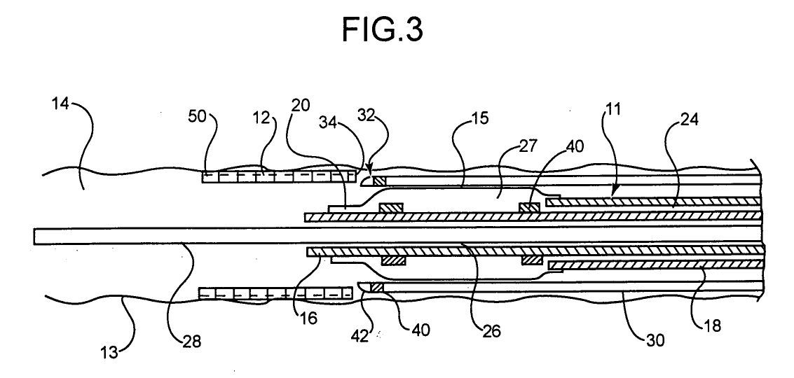

Once the device 10 has been advanced to a desired location within the vessel 14, the catheter 11 may be advanced within the sheath 30, such as in the manner shown in FIG. 2. As the catheter 11 is advanced the balloon 15 will push against the stent 12 to advance the stent out of the confines of the sheath 30. During advancement of the catheter 11, the balloon 15 may remain in the deflated state or be partially inflated as desired. In at least one embodiment the balloon 15 is inflated to a pressure of about 2 ATM. As the stent 12 exits the sheath 30 the stent 12 will expand to its expanded state within the vessel 14, such as is shown in FIG. 3.

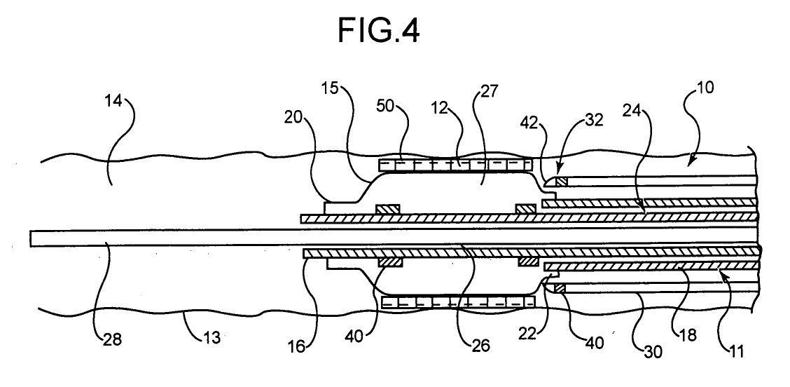

In some cases it may be necessary or desired to further expand the stent 12 within the vessel 14 in order to seat the stent 12 into the vessel wall 13. As is shown in FIG. 4, seating the stent 12 is accomplished by advancing the catheter 11 so that the balloon 15 is positioned under the stent 12 and then expanded to a desired pressure and diameter. The outward acting force supplied to the stent 12 by the expanding balloon 15 at least partially embeds or seats the stent 12 into the vessel wall 13.

Once the stent 12 is properly seated, the balloon 15 is deflated and the catheter 11 and sheath 30 may be withdrawn from the vessel 14. When the balloon 15 is evacuated of inflation fluid after seating the stent, the catheter 11 may be withdrawn back into the sheath 30. Deflation and/or refold of the balloon 15 may be aided by pulling the balloon 15 back into the confines of the sheath interior. In some embodiments the distal tip 42 of the sheath may be at least partially constructed of a material that has a softer durometer value than the rest of the sheath in order to better accommodate reinsertion of the balloon 15 therein. In some embodiments the tip 42 may be coated or otherwise configured to aid in reinsertion of the balloon 42 within the sheath 30.

In some embodiments the outer shaft 18 may have one or more coils and/or other implantable devices positioned proximal of the balloon 15. As such, if delivery of such devices is desired or necessary the balloon 15 may be fully advanced out of the sheath 30 to expose the coils for delivery.

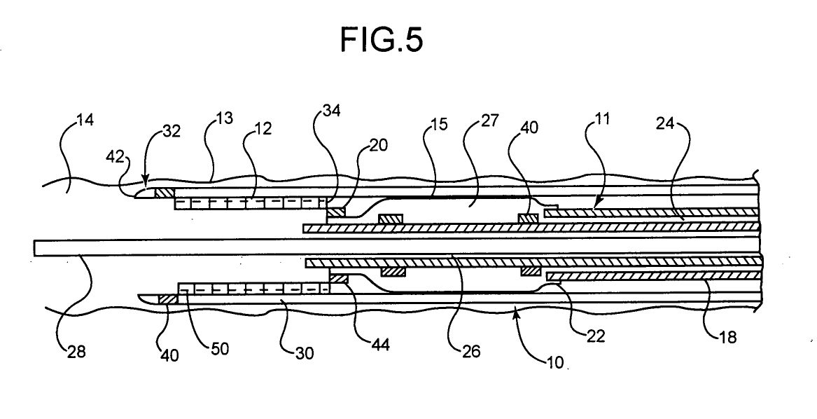

In at least one embodiment, such as in the example shown in FIG. 5, a portion of the balloon 15 adjacent to the stent 12 is provided with a reinforced and/or thicker region 44, which is configured to contact the stent 12 during advancement of the catheter 11 during stent deployment.

The reinforced region 44 may be at least a portion of the distal waist 20 of the balloon which has a thickness sufficient to engage the interior of the stent 12 and/or abut the proximal edge 34 of the stent during deployment. The reinforced region 44 may also comprise other portions of the balloon 15 that have been provided with one or more layers or coatings of material which are overlaid or otherwise engaged to the balloon to act as a interposing barrier between the balloon 15 and the edge 34 of the stent 12.

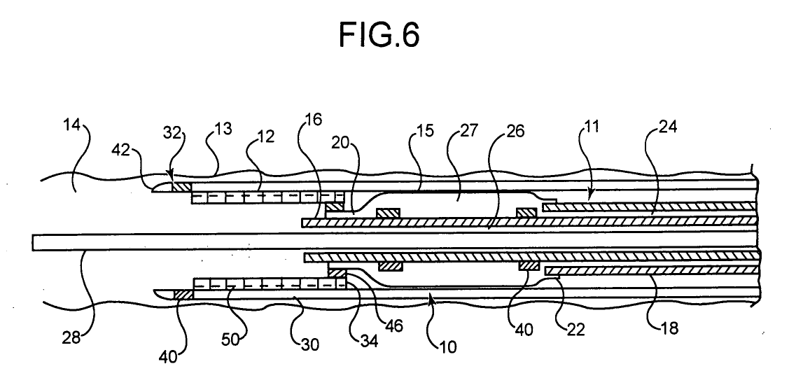

In at least one embodiment, such as in the example shown in FIG. 6, the catheter 11 may also/or alternatively employ at least one hub, ring, or other engagement member 46 which is positioned on the inner shaft 16 between the balloon 15 and stent 12. The engagement member 46, has a diameter and/or configuration designed to protect prevent direct contact between the balloon 15 and stent edge 34. An engagement member 46 may be constructed of a variety of materials and may be secured to the shaft 16 and/or balloon 15 in any of a variety of ways, including but not limited to: chemical and/or thermal welding, chemical adhesive, mechanical engagement, integral formation with the shaft, etc. Materials suitable for constructing the reinforcing region 44 and/or the engagement member 46 include but are not limited to: urethane, pebax, silicone, etc.

In some embodiments the device 10 may be configured to retrieve and/or re-sheath the stent 12 subsequent to delivery.

As a result of the unique arrangement of the stent 12 relative to the balloon 15, the device 10, including the sheath 30, is able to achieve an outer diameter or profile that is less than that of many other systems.

In the various embodiments shown and described the device 10 may also employ marker bands 40 to allow a practitioner to detect the position of the device 10 and/or its components as it/they are advanced and positioned within the vessel 14.

The phrase “marker bands” should be understood to represent any area, band, coating, member, etc. of the device 10 that is made to be radiopaque or otherwise detectable by imaging modalities such as X-Ray, MRI or ultrasound. In the embodiment shown in FIGS. 1-6 the distal region 32 of the outer sheath 30 comprises a marker band 40. Marker bands 40 are also positioned under the balloon 15, on the inner shaft 16. Such bands may alternatively or additionally placed on the stent 12, the balloon 15 or any other component of the device 10. For example, in the embodiment shown in FIG. 6, the engagement member 46 may comprise or be comprised of a marker band 40.

In some embodiments the stent 12 or other element of the device 10 may comprise one or more therapeutic agents. In some embodiments the agent is placed on the stent in the form of a coating 50. In at least one embodiment the coating 50 includes at least one therapeutic agent and at least one polymer agent.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the preferred and alternate embodiments of the invention. Those skilled in the art may recognize other equivalents to the specific embodiment described herein which equivalents are intended to be encompassed by the claims attached hereto.

1. A medical device comprising:
a catheter, the catheter having a balloon positioned thereon, the balloon having a reduced diameter state and an expanded diameter state;
a stent, the stent having a reduced diameter configuration, an expanded diameter configuration, and a seated diameter configuration, in the reduced diameter configuration the stent being disposed about a portion of the catheter distally adjacent to the balloon; and
a sheath, the sheath being disposed about the catheter and the stent in the reduced diameter configuration, the catheter being moveable relative to the sheath.

2. The medical device of claim 1 wherein the balloon comprises an outer diameter, and the stent comprises an inner diameter, when the balloon is in the reduced diameter state and the stent is in the reduced diameter configuration, the outer diameter of the balloon being at least as large as the inner diameter of the stent.

3. The medical device of claim 1 wherein when the stent is in the reduced diameter configuration the stent is positioned immediately adjacent to the balloon.

4. The medical device of claim 1 wherein the catheter comprises an inner shaft, the inner shaft defining a guidewire lumen for passage of a guidewire therethrough.

5. The medical device of claim 4 wherein the catheter comprises an outer shaft, the outer shaft disposed about a portion of the inner shaft, the balloon having a proximal waist portion and a distal waist portion, the proximal waist portion engaged to the outer shaft, the distal waist portion engaged to the inner shaft, the outer shaft and the inner shaft defining an inflation lumen therebetween, the inflation lumen in fluid communication with the balloon.

6. The medical device of claim 1 wherein the stent is a self-expanding stent.

7. The medical device of claim 6 wherein the stent is at least partially constructed of at least one shape memory material.

8. The medical device of claim 6 wherein when the sheath is disposed about the stent, the sheath retains the stent in the reduced diameter configuration.

9. The medical device of claim 1 wherein the stent comprises a therapeutic agent.

10. The medical device of claim 9 wherein the therapeutic agent comprises a coating of at least one pharmaceutical product on at least a portion of the stent.

11. The medical device of claim 10 wherein the coating comprises at least one polymer agent.

12. The medical device of claim 1 further comprising at least one radiopaque marker band, the at least one radiopaque marker band being positioned on at least one member of the group consisting of: the catheter, the sheath, the stent, and any combination thereof.

13. The medical device of claim 1 wherein the catheter comprises at least one stent engagement member, the at least one stent engagement member positioned between the balloon and the stent, the at least one stent engagement member having a diameter sufficient to engage at least a portion of the stent in the reduced diameter configuration.

14. The medical device of claim 1 wherein at least a portion of the balloon comprises a reinforced region, the reinforced region being immediately adjacent the stent in the reduced diameter configuration.

15. The medical device of claim 14 wherein the reinforced region comprises a coating of a reinforcing material positioned on at least the distal waist of the balloon.

16. The medical device of claim 14 wherein the reinforced region comprises a region of the balloon having a thickness greater than that of a remainder of the balloon.

17. A method of delivering a stent comprising the steps of:
providing a medical device, the medical device comprising:
a catheter, the catheter having a balloon positioned thereon, the balloon having a reduced diameter state and an expanded diameter state,
a stent, the stent having a reduced diameter configuration, an expanded diameter configuration, and a seated diameter configuration, in the reduced diameter configuration the stent being disposed about a portion of the catheter distally adjacent to the balloon, and
a sheath, the sheath being disposed about the catheter and the stent in the reduced diameter configuration, the catheter being moveable relative to the sheath;
advancing the medical device to a desired location in a body lumen;
advancing the catheter within the sheath, the balloon engaging a portion of the stent, wherein advancing the catheter results in advancing the stent distally out of the sheath; and
deploying the stent distally adjacent from the sheath and catheter within the lumen, the stent expanding from the reduced diameter configuration to the expanded diameter configuration.

18. The method of claim 17 further comprising the steps of:
advancing the catheter through the stent in the expanded configuration, wherein the stent is disposed about at least a portion of the balloon; and
expanding the balloon from the reduced diameter state to the expanded diameter state, the in expanded diameter state the balloon expanding the stent from the expanded diameter configuration to the seated diameter configuration.

19. The method of claim 18 further comprising the steps of:
reducing the balloon from the expanded diameter state to the reduced diameter; and
withdrawing the catheter, wherein the balloon is withdrawn back into the sheath in the reduced diameter state.
